(19) United States
(12) Patent Application Publication
(10) Pub. No.: US 2006/0089688 A1
(43) Pub. Date: Apr. 27, 2006

(54) Method and apparatus to reduce wrinkles through application of radio frequency energy to nerves

(76) Inventor: Dorin Panescu, San Jose, CA (US)

Correspondence Address: IRELL & MANELLA LLP, 840 NEWPORT CENTER DRIVE, SUITE 400, NEWPORT BEACH, CA, 92660, US

(21) Appl. No.: 10/973,170

(22) Filed: Oct. 25, 2004

(51) Int. Cl. A61F 2/00

(52) U.S. Cl. 607/101

(57) ABSTRACT

An apparatus and method for reducing wrinkles in the skin tissue of a patient. The method includes detecting a nerve. The nerve can be detected by inserting an electrode into the skin tissue, delivering a current and then observing any stimulation of muscle tissue. The process of inserting the electrode, delivering current and detecting muscle stimulation may be repeated at a plurality of different skin location to trace the nerve. Once the nerve is traced, the electrode can be inserted into the skin at various locations, either at or adjacent to the nerve, and deliver energy into the tissue at a level sufficient to disrupt interaction between the nerve and the adjacent muscle. Once the nerve/muscle interaction is disrupted the muscle will be relaxed and the wrinkle(s) will either be removed or reduced.

### BACKGROUND OF THE INVENTION

1. Field of the Invention

The present invention relates to a method and apparatus for treating wrinkled skin tissue.

2. Prior Art

Radio frequency energy has been used in various medical procedures. For example, radio frequency probes have been used to cauterize and coagulate blood. Radio frequency energy has also been used in ophthalmic procedures.

Refractec, Inc. of Irvine, Calif., the assignee of the present application, has developed a system to correct hyperopia and presbyopia with a probe that is connected to a console. The probe includes a tip that is inserted into the stroma layer of a cornea. Electrical current provided by the console flows through the eye to denature the collagen tissue within the stroma. The process of inserting the probe tip and applying electrical current can be repeated in a circular pattern about the cornea. The circular band of the denatured areas decreases the radius of the eye. The procedure of applying RF energy through a probe tip to denature corneal tissue is taught by Refractec under the service marks CONDUCTIVE KERATOPLASTY and CK.

Thermage, Inc. of Hayward, Calif. has marketed a product that “removes” wrinkles through the application of radio frequency energy. Thermage has also obtained a number of related patents, including U.S. Pat. Nos. 6,749,624; 6,470,216; 6,461,378; 6,453,202; 6,438,424; 6,430,446; 6,425,912; 6,413,255; 6,405,090; 6,387,380; 6,381,498; 6,381,497; 6,377,855; 6,377,854; 6,350,276; 6,311,090; 6,241,753; 5,948,011; 5,919,219; 5,871,524; and 5,755,753. To prevent overheating of the outer layer of skin tissue the Thermage product incorporates a cooling system that creates a reverse thermal gradient through the skin. The cooling system requires a heat exchanger, coolant lines, etc. that increase both the costs and complexity of the system.

VNUS Medical, Inc. of San Jose, Calif. markets a product that treats venous diseases through the application of RF energy. VNUS also obtained a number of related patents, including U.S. Pat. Nos. 6,638,273; 6,613,045; 6,401,719; 6,398,780; 6,361,496; 6,322,559; 6,263,248; 6,258,084; 6,237,606; 6,231,507; 6,200,312; 6,179,832; 6,165,172; 6,152,899; 6,149,660; 6,139,527; 6,135,997; 6,071,277; 6,036,687; 6,033,398; 6,033,397; 6,014,589; 5,810,847; 5,730,136; and 5,609,598. The VNUS products are catheters that are inserted into the body. Such products are not practical for application to skin tissue.

In “Application of Radiofrequency Treatment in Practical Pain Management: State of the Art”, J. Van Zundert, P. Raj, S. Erdine, M. van Kleef, World Institute of Pain 2002, the authors discuss applications of radiofrequency treatment for various ailments such as chronic cervical, lower back, and intractable cancer pain.

There has been developed a product sold under the trademark BOTOX which injects small amounts of butulinum toxin A into the skin. The butulinum toxins attach themselves to nerve endings. Once this happens, acetylcholine, the neurotransmitter for triggering muscle contractions, cannot be released. The end result is the disruption of the interaction between the nerves and the adjacent muscle tissue. The disruption causes the muscle to relax and eliminates or reduces wrinkles in the skin. The effects of BOTOX are temporary, typically only lasting a few months. It would be desirable to provide a procedure for reducing wrinkles that is safe, economical and has a more lasting effect.

### BRIEF SUMMARY OF THE INVENTION

A method and apparatus for reducing wrinkles in the skin tissue of a patient. The method includes detecting a nerve in the skin tissue and delivering energy into the tissue to disrupt the interaction between the nerve and muscle tissue.

### BRIEF DESCRIPTION OF THE DRAWINGS

FIG. 1 is a perspective view of a system for treating skin tissue;

FIG. 2 is a schematic of a console of the system;

FIG. 3 is a graph showing a waveform that is provided by a console of the system;

FIG. 4 is an enlarged view of a tip inserted into skin tissue;

FIG. 5 is an illustration showing the temporal, zygomatic and buccal nerves in a human face;

FIG. 6 is an alternate embodiment of a probe;

FIG. 7 is an alternate embodiment of a probe inserted into skin tissue;

FIG. 8 is an alternate embodiment of a probe;

FIG. 9 is an alternate embodiment of a probe;

FIG. 10 is an illustration showing a patient with a variety of different ground elements.

### DETAILED DESCRIPTION

Disclosed is an apparatus and method for reducing wrinkles in the skin tissue of a patient. The method includes detecting a nerve. The nerve can be detected by inserting an electrode into the skin tissue, delivering a current and then observing any stimulation of muscle tissue. Such stimulation may be in the form of a muscle twitch. The process of inserting the electrode, delivering current and detecting muscle stimulation may be repeated at a plurality of different skin location to trace the nerve.

Once the nerve is traced, the electrode can be inserted into the skin at various locations, either at or adjacent to the nerve, and deliver energy into the tissue at a level sufficient to disrupt interaction between the nerve and the adjacent muscle. The energy may damage the nerve or the nerve/muscle interface. The damage may either be permanent or temporary. Once the nerve/muscle interaction is disrupted the muscle will be relaxed and the wrinkle(s) will either be removed or reduced.

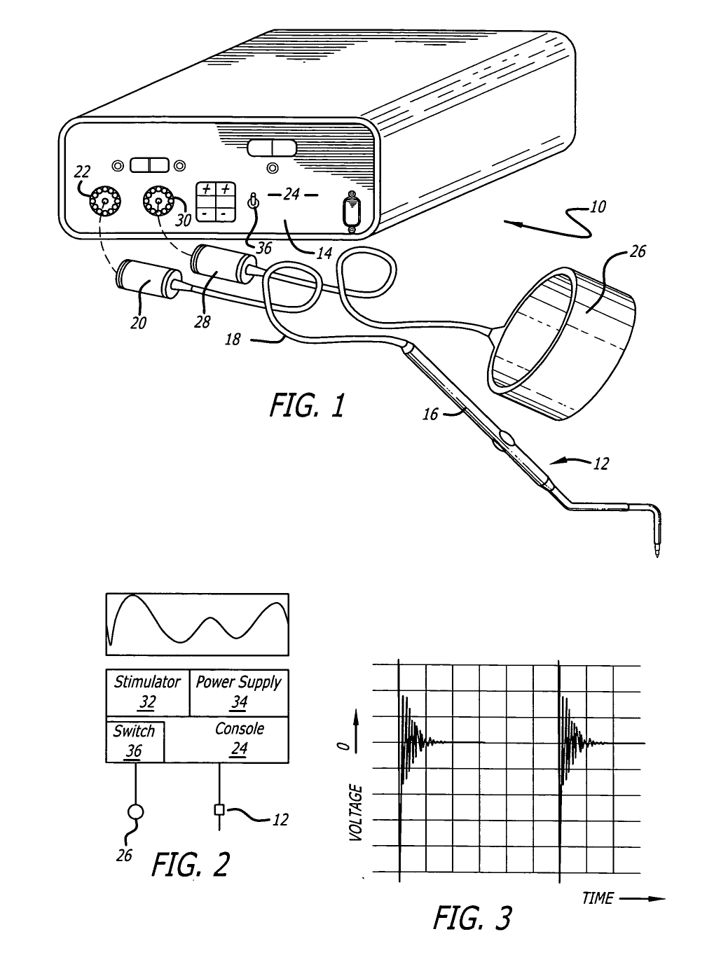

Referring to the drawings more particularly by reference numbers, FIG. 1 shows an embodiment of an apparatus 10 that can be used to reduce wrinkles. The apparatus 10 includes an electrode probe 12 coupled to a console 14. The probe 12 has a hand piece 16 and wires 18 that couple the probe electrode to a connector 20 that plugs into a mating receptacle 22 located on the front panel 24 of the console 14. The hand piece 16 may be constructed from a non-conductive material.

The system 10 also includes a return element 26 that is in contact with the patient to provide a return path for the electrical current provided by the console 14 to the probe 12. The return element 26 has a connector 28 that plugs into a mating receptacle 30 located on the front panel 24 of the console 14. By way of example, the return element 26 may be a wrist band as shown, a bar, a pad, a ring, a patch or any other configuration to create a relatively low impedance contact with the patient.

As shown in FIG. 2, the console 14 may contain a neuro-stimulator 32 and a power supply 34 that can deliver electrical power to the probe 12. In general, the neuro-stimulator 32 can provide energy to stimulate a nerve and the power supply 34 can provide energy to disrupt interaction between the nerve and muscle tissue. The console 14 may have a switch 36 which allows the user to switch between the stimulator 32 and the power supply 34. Although the neuro-stimulator 32 and power supply 34 are shown in the same console, these elements 32 and 34 could be separate devices, not necessarily built within the same enclosure. Additionally, the stimulator 32 and the power supply 34 can be used as part of one procedure, or as part of separate procedures (e.g. nerve tract identification procedure and wrinkle reduction procedure).

The power supply 32 may provide a predetermined amount of energy, through a controlled application of power for a predetermined time duration. The power supply 32 may have manual controls that allow the user to select treatment parameters such as the power and time duration. The power supply 32 can also be constructed to provide an automated operation. The power supply 32 may have monitors and feedback systems for measuring physiologic tissue parameters such as tissue impedance, tissue temperature and other parameters, and adjust the output power of the radio frequency amplifier to accomplish the desired results.

In one embodiment, the power supply 32 provides voltage limiting to prevent arcing. To protect the patient from overvoltage or overpower, the power supply 32 may have an upper voltage limit and/or upper power limit which terminates power to the probe when the output voltage or power of the unit exceeds a predetermined value.

The power supply 32 may also contain monitor and alarm circuits which monitors physiologic tissue parameters such as the resistance or impedance of the load and provides adjustments and/or an alarm when the resistance/impedance value exceeds and/or falls below predefined limits. The adjustment feature may change the voltage, current, and/or power delivered by the console such that the physiological parameter is maintained within a certain range. The alarm may provide either an audio and/or visual indication to the user that the resistance/impedance value has exceeded the outer predefined limits. Additionally, the unit may contain a ground fault indicator, and/or a tissue temperature monitor. The front panel 24 of the console 14 typically contains meters and displays that provide an indication of the power, frequency, etc., of the power delivered to the probe.

The power supply 32 may deliver a radiofrequency (RF) power output in a frequency range of 100 KHz-5 MHz. In the preferred embodiment, power is provided to the probe at a frequency in the range of 350 KHz. The console 14 is designed so that the power supplied to the probe 12 does not exceed a certain upper limit of up to 50 watts. The time duration of each application of power to a particular location of tissue can be up to several seconds.

If the system incorporates temperature sensors, the power supply 32 could control the power such that the target skin tissue temperature is maintained to no more than 85° C., to avoid necrosis of the tissue. The temperature sensors can be carried by the probe 12, incorporated into the electrodes 40, or attached within proximity to the electrodes 40.

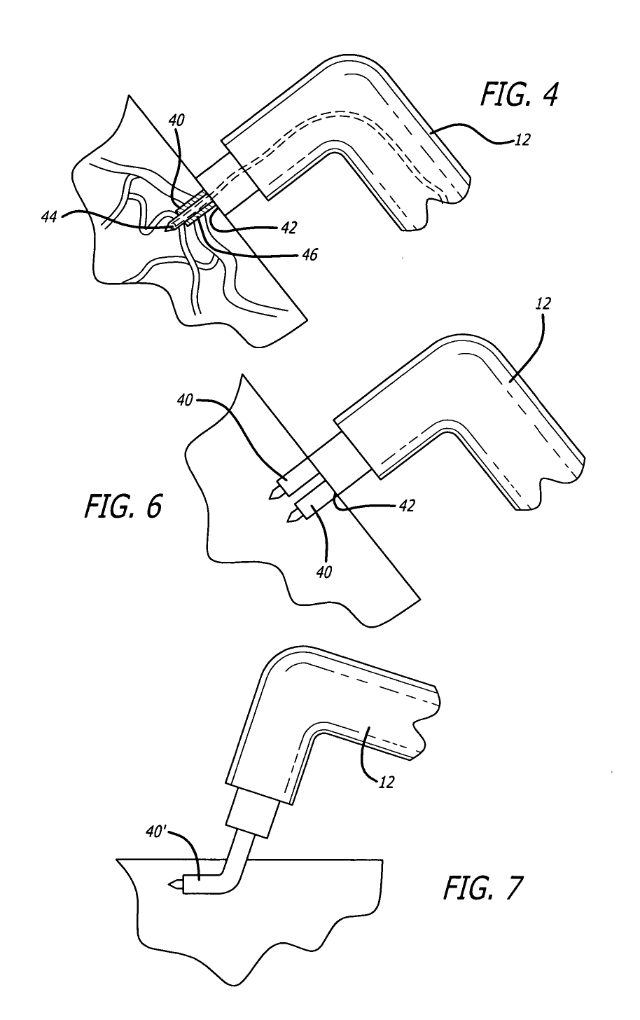

If the system includes an impedance monitor, the power could be adjusted so that the target tissue impedance, assuming a probe 12 with a tip of length 460 um and diameter of 90 um, decreases by approximately 50% from an initial value that is expected to range between 2500 to 4000 ohm. The power supply 32 could regulate the power down if, after an initial descent, the impedance begins to increase. Controls can be incorporated to terminate RF delivery if the impedance increases by a significant percentage from the baseline. Alternatively, or additionally, the power supply 32 can modulate the duration of RF delivery such that delivery is terminated only when the impedance exceeds a preset percentage or amount from a baseline value, unless an upper time limit is exceeded. Other time-modulation techniques, such as monitoring the derivative of the impedance, could be employed. Time-modulation could be based on physiologic parameters other than tissue impedance (e.g tissue water content, chemical composition, etc.) FIG. 3 shows a typical voltage waveform that is delivered by the probe 12 to the skin. Each pulse of energy delivered by the probe 12 may be a highly damped sinusoidal waveform, typically having a crest factor (peak voltage/RMS voltage) greater than 5:1. Each highly damped sinusoidal waveform is repeated at a repetitive rate. The repetitive rate may range between 4-12 KHz and is preferably set at 7.5 KHz. Although a damped waveform is shown and described, other waveforms, such as continuous sinusoidal, amplitude, frequency or phase-modulated sinusoidal, etc. can be employed.

FIG. 4, shows an embodiment of a probe 12 with a tip 40 that is inserted into skin tissue. The probe 12 may have a stop 42 that limits the insertion depth of the tip 40. Although a stop 42 is shown and described, it is to be understood that the probe 12 does not need to have a stop 42. The stop 42 may be constructed from an insulator material to minimize the flow of current on the top layer of skin.

The tip 40 should have a length that insures sufficient penetration into the skin to reach a nerve. By way of example, the tip 40 may have a length between 500 to 2500 microns, with a preferred length of 1500 microns.

The diameter of the tip 40 should be sufficient to provide the desired amount of energy but be small enough to not leave unsightly incision wounds. The tip may have a diameter of 50 to 200 microns, with a preferred diameter of 150 microns. The tip 40 could carry, have embedded in it, or otherwise attached to it, specialized sensors 44, such as temperature sensors (e.g. thermocouples, thermistors, etc.), pressure sensors, etc. Although specific lengths and diameters have been disclosed, it is to be understood that the tip may have different lengths and diameters. A portion of the tip 40 may be insulated with a non-conductive insulator 46. The insulator 46 may insure that current only flows from the distal end of the tip to provide a localized energy application.

In operation, the treated skin is typically washed and then wiped with alcohol. The probe tip 40 is inserted into the skin and then current is delivered from the neuro-stimulator 34 to the tissue, if the nerve pacing is needed in order to establish the trajectory of the targeted nerve bundle. The neuro-stimulator current may be provided in pulses having a duration up to 20 milliseconds at a frequency up to 50 Hz. The tip may provide a nerve-pacing current up to 10 milliamps. The tip is inserted and energy is delivered until a stimulation of muscle tissue is detected indicating a nerve location. The stimulation of muscle tissue may be in the form of a muscle twitch. The process of inserting the tip, delivering energy and detecting muscle stimulation is repeated at a number of skin location to trace the nerve.

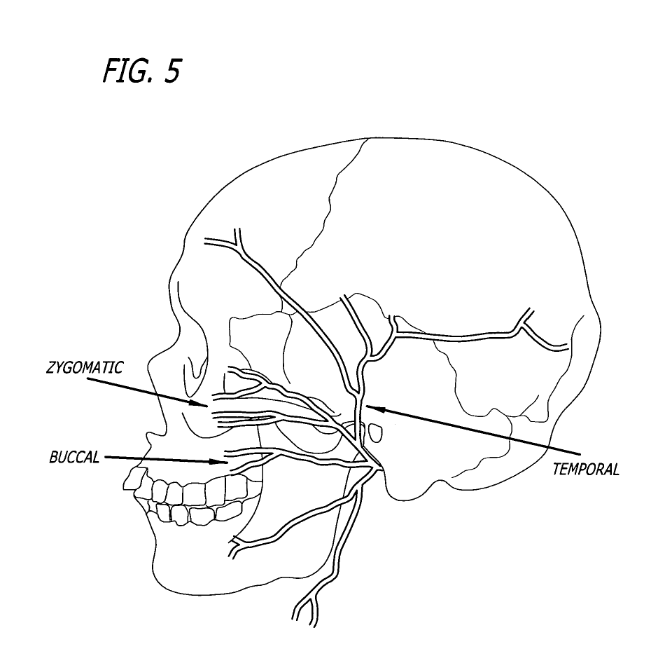

It is desirable to monitor the reaction of collateral structures during stimulation. The user should preferably avoid locations that can stimulate other nerve or muscles structures not targeted for the wrinkle reduction procedure. Additional steps of identifying the location of collateral nerves or muscles could be taken, if necessary. For facial wrinkles it is desirable to detect the temporal, zygomatic and/or buccal nerves shown in FIG. 5. The ophthalmic nerve should be avoided.

Once the nerve has been traced, the tip 40 can be reinserted into the skin to deliver energy sufficient to disrupt the interaction between the nerve and the muscle tissue. The process of inserting the tip and delivering energy to disrupt the nerve/muscle interaction can be repeated along the nerve. The tip may be applied directly at the nerve, or at the nerve/muscle interface. The energy may permanently or temporarily disrupt the interaction between the nerve and the adjacent muscle tissue. The energy should not exceed 50 watts.

The pacing step could then be repeated to ensure that conduction to the targeted muscle has been severed. The procedure may be iterated several times until the desired effect is reached. It is desirable to start with a low energy level to ensure that the nerve damage is controlled. The target structure could be the nerve branch itself or the nerve-muscle interface. Damage to the nerve branch could cease propagation of nervous impulse conduction along the axon. Damage to the nerve-muscle interface would preclude receptors on the muscle side to respond to impulses transmitted by the nerve.

The damage could be permanent or temporary, depending on the level of energy required. In certain cosmetic procedures, longer-term temporary severance of nerve-muscle communication might be preferred over permanent severance. Once the nervous impulse propagation to the targeted muscle structure is inhibited, temporarily or permanently, the muscle becomes relaxed. In the short period of time, the appearance of the associated skin wrinkles will be reduced or will disappear.

Following the procedure the patient may be given oral minocycline, 100 milligrams, every 12 hours for 4 days. The treated area may also be regularly washed with antiseptic soap and water.

FIG. 6 shows an alternate embodiment of a probe that has a pair of tips 40. The probe may be a bi-polar device wherein one of the tips is a return ground element. Alternatively, combined bi-polar/monopolar energy delivery could be employed. In such case, an indifferent return electrode could be used in conjunction with a bi-polar probe. The use of a bi-polar probe may create a flow of current that is essentially parallel with the surface of the skin. Bi-polar energy applications have the advantage of confining treatment to better-specified boundaries. Such bi-polar probes could be used in situations when the target treatment zone is limited in size.

FIG. 7 shows another embodiment of a probe with a bent tip 40′. The bent tip 40′ may be inserted into the skin in a manner so that the tip is essentially parallel with the skin surface. The probe may have a pair of bent tips 40′ so that the tips 40′ create a localized flow of current.

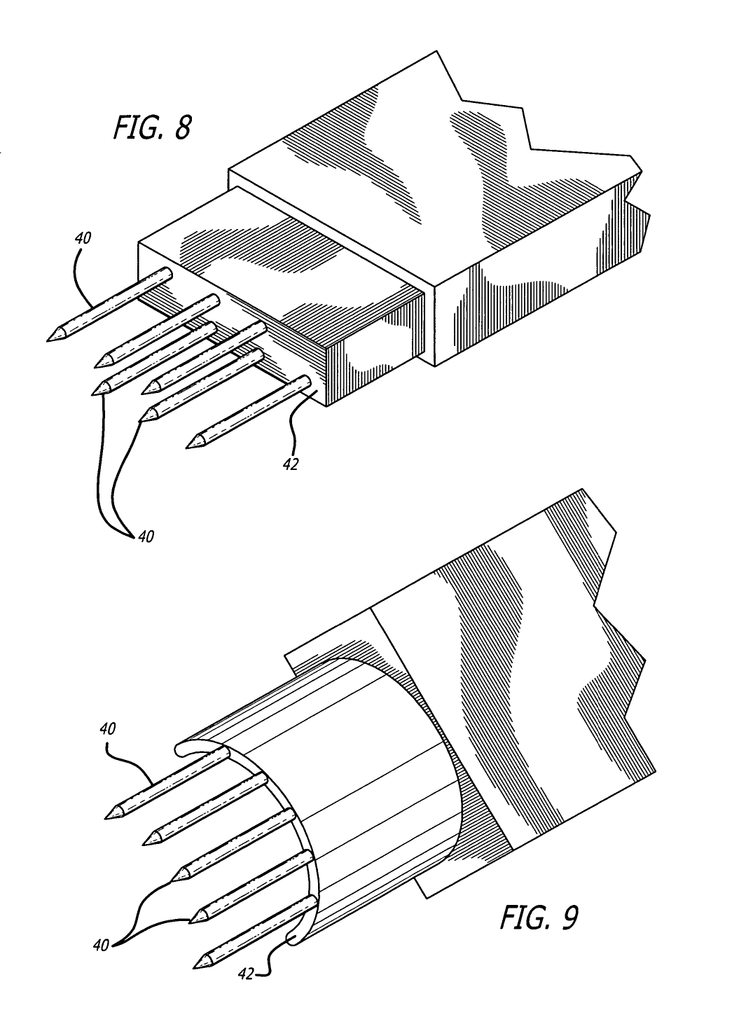

FIG. 8 shows another embodiment of a probe with a plurality of tips 40. The tips 40 may be connected to the console so that there are a number of bi-polar tip pairs. This embodiment allows for the simultaneous delivery of energy, thereby reducing the time required to perform a procedure.

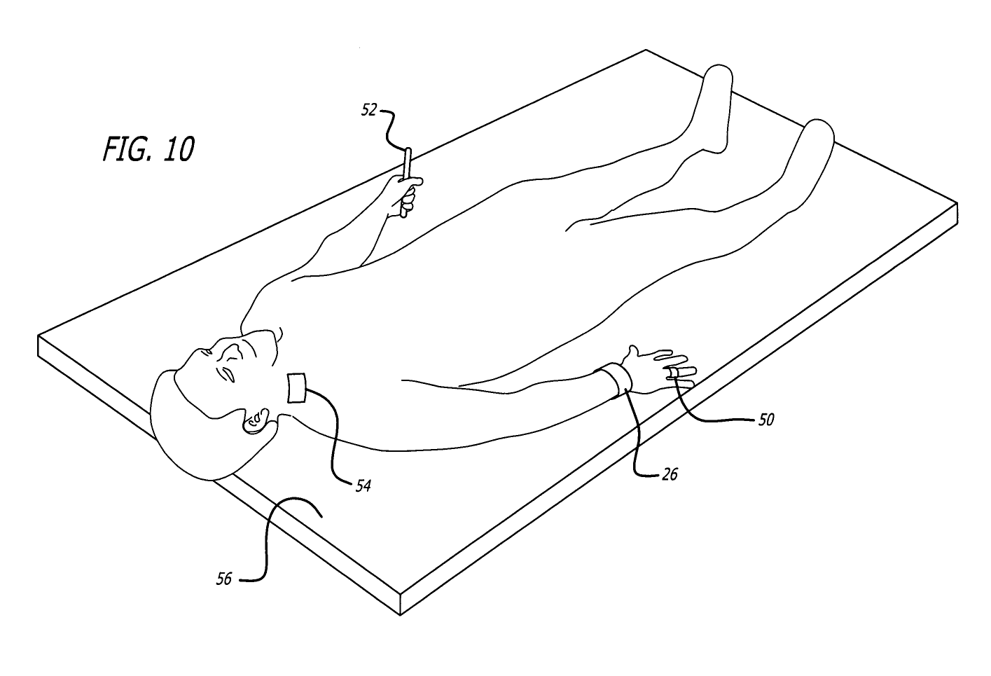

FIG. 9 shows another embodiment of a probe with a plurality of tips 40 arranged in a pattern. This probe may also allow for the simultaneous delivery of energy to reduce the time required to perform a procedure.

FIG. 10 shows the use of different ground elements 26, 50, 52, 54 and 56. The ground element could be configured as a ring 50 worn on the patient's finger, a wristband 26 wrapped around the patient's wrist or a rod 52 held by the patient. The ground element may be configured as a patch 54 that is attached to the patient by an adhesive or a plate 56 placed under the patient. Although the plate 56 is shown as being larger than the patient, the plate 56 may have different sizes and shapes.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

For example, although the delivery of radio frequency energy is described, it is to be understood that other types of non-thermal energy such as direct current (DC), microwave, ultrasonic and light can be transferred into the skin tissue through the probe.

By way of example, the console can be modified to supply energy in the microwave frequency range or the ultrasonic frequency range. By way of example, the probe may have a helical microwave antenna with a diameter suitable for delivery into the tissue. The delivery of microwave energy could be achieved with or without tissue penetration, depending on the design of the antenna. The system may modulate the microwave energy in response to changes in the characteristic impedance.

For ultrasonic application, the probe would contain a transducer that is driven by the console and mechanically oscillates the tip. The system could monitor acoustic impedance and provide a corresponding feedback/regulation scheme. For application of light the probe may contain some type of light guide that is inserted into the skin and directs light into the tissue. The console would have means to generate light, preferably a coherent light source, such as a laser, that can be delivered by the probe. The probe may include lens, waveguide and a photodiode that is used sense reflected light and monitor variations in the index of refraction, birefringence index of the tissue as a way to monitor physiological changes and regulate power.

Although the methods and apparatuses described above employ probes that are inserted into the skin to reach in to nerve or muscle bundles, one ordinarily skilled in the art could develop equivalent techniques and equipment that do not require skin penetration by the energy-delivering electrode. For example, focused-ultrasound can be used to focus ultrasound waves, emitted from a transmitter that resides on the surface of the skin, into the targeted nerve or muscle bundles. Similarly, while the disclosure above presents use of the same probe for nerve tracing and for the disruption of the nerve-muscle interaction, separate probes of different designs can be used. Nerve tracing can also be performed by employing other conventional techniques such as imaging, stimulation with existing equipment, etc.

What is claimed is:

1. A method for reducing wrinkles in a skin tissue of a patient, comprising:
locating a nerve structure in a skin tissue; and,
delivering energy into the skin tissue with a probe to disrupt the interaction between the nerve structure and muscle tissue.

2. The method of claim 1, wherein the energy includes electrical current in the radio frequency spectrum.

3. The method of claim 1, wherein the energy includes a direct current.

4. The method of claim 1, wherein the energy includes energy in the microwave spectrum.

5. The method of claim 1, wherein the energy includes energy in the ultrasound spectrum.

6. The method of claim 1, wherein the energy includes optical energy.

7. The method of claim 1, wherein the energy is less than 50 watts.

8. The method of claim 1, wherein the probe includes an electrode inserted into the skin tissue.

9. The method of claim 1, further comprising repeating the process of delivering energy to disrupt the interaction between the nerve structure and the muscle tissue at a plurality of skin tissue locations.

10. The method of claim 1, wherein the energy delivered permanently disrupts the interaction between the nerve structure and muscle tissue.

11. The method of claim 1, wherein the energy delivered temporarily disrupts the interaction between the nerve structure and muscle tissue.

12. The method of claim 1, wherein the nerve structure is located by inserting a probe into the skin tissue, delivering stimuli, and detecting a muscle response.

13. The method of claim 12, wherein the stimuli includes a current.

14. The method of claim 12, further comprising repeating the process of inserting the probe, delivering stimuli, and detecting a muscle response at a plurality of skin tissue locations.

15. A system for reducing wrinkles in a skin tissue of a patient, comprising:
a probe; and,
a power supply connected to said probe, said power supply provides energy to disrupt the interaction between a nerve structure and muscle tissue.

16. The system of claim 15, wherein said probe includes a first electrode.

17. The system of claim 16, wherein said first electrode includes a stop.

18. The system of claim 16, wherein said first electrode includes an insulated portion and a non-insulated tip.

19. The system of claim 16, wherein said probe includes a second electrode.

20. The system of claim 15, wherein said power supply provides a current in a radio frequency spectrum.

21. The system of claim 15, wherein the energy includes a direct current.

22. The system of claim 15, wherein the energy includes energy in the microwave spectrum.

23. The system of claim 15, wherein the energy includes energy in the ultrasound spectrum.

24. The system of claim 15, wherein the energy includes optical energy.

25. The system of claim 15, wherein said power supply provides stimuli to detect the nerve structure.
